(19) United States
(12) Patent Application Publication
(10) Pub. No.: US 2023/0263808 A1
(43) Pub. Date: Aug. 24, 2023

(54) THERAPEUTIC USES OF GLUCOCORTICOIDS WITH ANABOLIC EFFECTS IN SKELETAL MUSCLE

(71) Applicant: UNIVERSITY OF FLORIDA RESEARCH FOUNDATION, INCORPORATED, Gainesville, FL (US)

(72) Inventors: Kendall Nettles, Gainesville, FL (US); Nelson Bruno, Gainesville, FL (US); Theodore Kamenecka, Gainesville, FL (US); Glenn Micalizio, Gainesville, FL (US)

(21) Appl. No.: 18/007,308

(22) PCT Filed: Oct. 13, 2021
(86) PCT No.: PCT/US2021/054804

§ 371 Date: Jan. 30, 2023

Related U.S. Application Data
(60) Provisional application No. 63/092,684, filed on Oct. 16, 2020.

(51) Int. Cl. A61K 31/58; A61P 21/00; A61P 29/00

(52) U.S. Cl. CPC A61K 31/58; A61P 21/00; A61P 29/00

(57) ABSTRACT

The present invention provides methods for treating and ameliorating symptoms of skeletal muscle cachexia and related conditions, including muscle wasting and muscle weakness.

### CROSS-REFERENCE TO RELATED APPLICATIONS

The subject patent application claims the benefit of priority to U.S. Provisional Patent Application No. 63/092,684 (filed Oct. 16, 2020; now pending). The full disclosure of the priority application is incorporated herein by reference in its entirety and for all purposes.

### BACKGROUND OF THE INVENTION

Glucocorticoids (GCs) are the most prescribed anti-inflammatory agents. They are frequently used for the treatment of acute and chronic inflammatory diseases, e.g. asthma, rheumatoid arthritis, inflammatory bowel diseases, multiple sclerosis and atopic dermatitis. GCs are also included with other agents in cancer treatment. The main mechanism by which GCs mediate anti-inflammatory activity is the transrepression of genes coding for cytokines (e.g., TNF-α and IL-6), adhesion molecules and enzymes involved in inflammation processes. However, treatment with GCs is also associated with undesired side effects. For example, GCs can induce insulin resistance and reduce glucose uptake in the primary organ for glucose disposal, skeletal muscle, and facilitate muscle wasting. These undesired effects are due to a combination of reduced protein synthesis and increased protein degradation. Efforts to develop improved glucocorticoids have been hampered by an almost complete lack of understanding of the chemical and molecular mechanisms through which glucocorticoid receptor (GR) ligands can differ in these phenotypic outcomes. This phenomenon is called selective modulation or dissociated signaling, and is a common feature of allosteric signaling for the nuclear receptor (NR) superfamily of transcription factors, such as GR, and for GPCRs and other allosteric drug targets. Understanding mechanisms through which ligands control different phenotypic outcomes is the single greatest barrier to developing improved selective modulators of NRs, GPCRs and other allosteric drug targets.

There is a need in the art for dissociated glucocorticoid compounds with better therapeutic activities and minimized side effects. The present invention is directed to this and other needs.

### SUMMARY OF THE INVENTION

In one aspect, the invention provides methods for treating an inflammatory condition or ameliorating symptoms of undesired inflammation in a subject. The methods involve administering to the subject in need of treatment a pharmaceutical composition comprising a therapeutically effective amount of a compound of Formula I below.

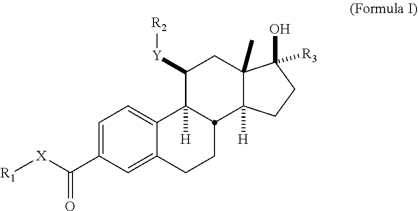

In Formula I, X and Y are each independently O or NR; R₁=aryl, substituted aryl, pyridine, CN, OR, halogen, or Rn; R₂=H, C1-C3 alkyl or alkenyl, aryl, heteroaryl, halo, benzyl, halogen, CN, OR, or Rn; R₃=C1-C3 alkyl, alkenyl, alkynyl, or Rn; wherein n=0-3, R=H, C1-C4 alkyl, or haloalkyl.

In some of the methods, the administered compound is anabolic. In some methods, the subject to be treated is afflicted with an inflammatory disorder or an autoimmune disease. Some methods employ a compound of Formula I wherein if X=NH, Y=O, R₁=pyridine, then R₂ is not methyl, ethyl, benzyl, 3-chlorobenzyl, 4-chlobenzyl or carbonate. Some methods of the invention employ a compound of Formula I wherein X=NH, Y=O, R₂=H, R₃=

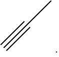

In some of these methods, the administered compound is compound 15418, 15420, 15438, 15419, 15421, 15439 or 11461 shown in FIG. 13. Some methods of the invention employ a compound of Formula I wherein X=NH, Y=O, R₁=pyridine, R₂=H. In some of these methods, the administered compound is compound 15480, 11464, 11465 or 15481 shown in FIG. 13. Some methods of the invention employ a compound of Formula I wherein X=NH, Y=O, R₁=pyridine, R₃=

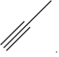

In some of these methods, the administered compound is compound 15960, 15961, 11466, 11469, 16024, 16023, 16025 or 14274 shown in FIG. 13. Some methods of the invention employ compound 16022 or 15918 shown in FIG. 13.

In some embodiments, the subject is also administered with an agent for chemotherapy in addition to the compound of Formula I. In some of these methods, the agent for chemotherapy is administered to the subject prior to, simultaneously with or subsequent to administration with the compound.

Some methods of the invention are directed to treating a subject that is afflicted with cancer. In various embodiments, the compound administered to the cancer patient is any of the compounds shown in FIG. 13. In some specific embodiments, the administered compound is compound 11466 or 16024 shown in FIG. 13.

In a related aspect, the invention provides methods for treating a muscular dystrophy or cachexia in a subject. These methods entail administering to the subject with a pharmaceutical composition comprising a therapeutically effective of a compound of Formula I below.

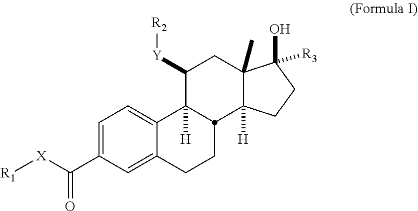

In Formula I, X and Y are each independently O or NR; R₁=aryl, substituted aryl, pyridine, CN, OR, halogen, or Rn; R₂=C1-C3 alkyl or alkenyl, aryl, heteroaryl, halo, benzyl, halogen, CN, OR, or Rn; R₃=C1-C3 alkyl, alkenyl, alkynyl, or Rn; wherein n=0-3, R=H, C1-C4 alkyl, or haloalkyl.

In some methods, the compound of Formula I that is administered to the subject is anabolic. In some methods, the administered compound is a compound of Formula I wherein if X=NH, Y=O, R₁=pyridine, then R₂ is not methyl, ethyl, benzyl, 3-chlorobenzyl, 4-chlobenzyl or carbonate. In various methods, the administered compound is any of the compounds shown in FIG. 13. In some specific embodiments, the administered compound is compound 11466 or 16024 shown in FIG. 13. In some embodiments, the subject to be treated is afflicted with Duchenne's muscular dystrophy.

A further understanding of the nature and advantages of the present invention may be realized by reference to the remaining portions of the specification and claims.

### BRIEF DESCRIPTION OF THE DRAWINGS

FIG. 1 shows chemical and structural design of glucocorticoids. A) Chemical structure of the dissociated glucocorticoid, PF802. B) Dexamethasone bound to the GR ligand binding domain (LBD). For the chemical structure, carbons 3,11, and 17 are indicated (1M2Z.pdb). C-E) Structures of the GR LBD bound to the indicated compounds with highlighted parts (PDBs: 1NHZ, 4P6W, and 3BQD). F) Model of SR11466 bound to the GR LBD.

FIG. 2 shows quantitative phenotyping assays for GC action in skeletal muscle. A-E) Myotubes were nutrient-deprived, pre-treated with DMSO, RU486, or Dex, and treated with insulin as outlined in FIG. 7. A) C2C12 myotubes were assayed for chymotrypsin-like proteasomal activity. B) pAKT in C2C12 myotubes was compared by In-Cell Western assay (ICW) 48 h after treatment with RU486 or Dex. C) Quantitation of the ICW in panel B. D) ICW for surface expression of Glut4 on L6 myotubes. E) ICW for insulin-induced incorporation of puromycin into C2C12 myotube surface proteins. F-G) High-content imaging and analysis of C2C12 myoblasts stained with a Mitotracker dye. H) Assay reproducibility from screening 22 compounds on two separate occasions. The Pearson correlation coefficient, r, and its associated p-value is indicated.

FIG. 3 shows correlation between variables reveals interconnected signaling networks. A) Pearson correlations were calculated between the indicated variables. The different lines indicate statistically significant correlations at the corresponding p-value thresholds. Solid lines indicate positive correlations, dashed lines indicate negative correlations. B-E) Correlations between the effects of all tested compounds in the indicated assays. Each data point represents the mean effects of a distinct compound. The Pearson correlation coefficient, r, and associated p-values are indicated. F) pAKT levels in C2C12 myotubes assayed 1 h after Dex treatment. G) Significant Pearson correlations for the C11-substituted compounds. H) Correlations between the effects of C11-substituted compounds in the indicated assays.

FIG. 4 shows GR-target genes and -interacting peptides predict GC function in skeletal muscle. A-D) GC-regulated predictors of Glut4 translocation, protein synthesis, mitochondrial potential, and pAKT in the sets of all tested compounds and C11-substituted compounds. E-H) Correlations between the effects of C11-substituted compounds on the indicated mRNA levels (y-axes) and GC-regulated phenotypes (x-axes). I) Correlations between the effects of C11-substituted compounds on GR interaction with the indicated peptides (y-axes) and GC-regulated phenotypes (x-axes).

FIG. 5 shows machine learning reveals best predictors of pathway-specific signaling. A) Predictors of GC-regulated phenotypes in the set of all tested compounds (Dataset S1) were identified using Boruta. For each phenotype, a minimal set of predictors is also highlighted. B-E) The effects of all tested compounds in the indicated assays. Each data point represents the mean effects of a distinct compound.

FIG. 6 shows functional validation of the predictive target gene, Fkbp5, and GR coregulators. A-C) Mice were electroporated with either empty vector (GFP) control and either Fkbp5 or Foxo1 expression plasmids in contralateral Tibias Anterior (TA) muscles. After 7 days, the transduced mice were fasted overnight and treated with insulin for 1 hr. A) Weight of transduced TA muscles. B-C) Western blot and quantitation of insulin-induced B) puromycin incorporation i.e. protein synthesis and C) pAKT in transduced TA muscles. D) Effect of Dex on insulin-induced protein synthesis in C2C12 myotubes transduced with lentivirus expressing the indicated shRNAs. E) The effects of all tested compounds on insulin-induced protein synthesis (x-axes) and GR interactions with the indicated peptides. F) Effect of Dex on insulin-induced pAKT in C2C12 myotubes transduced with lentivirus expressing the indicated shRNAs. G) The effects of all tested compounds on insulin-induced pAKT (x-axes) and GR interactions with the indicated peptides.

FIG. 7 shows glucocorticoids with improved skeletal muscle profiles. A) Correlation of correlation (COC) plots where each point represents the Pearson correlation coefficient, r, between a distinct gene expression or GR-peptide interaction assay with assays indicated on the x and y axes. B) The effects of all tested compounds on IL-1β-induced secretion of IL-6 (y axes) and GC-regulated phenotypes indicated on the x axes. C) The effects of all tested compounds on IL-1β-induced secretion of IL-6 (y axis) and bone mineralization (x axis). D) IL-1β-induced IL-6 production by A549 cells treated with 10 μM of the indicated compounds was compared by AlphaLISA. E-H) Myoblasts were assayed as described in FIG. 8. I) Representative myoblasts imaged during the mitochondrial potential assay. J) Plasma TNFα levels of C57BL/6 male mice treated pretreated with the indicated ligands. K) Changes in lean mass of mice in panel J were determined by whole-body NMR after an additional 18 h LPS treatment. Data shown are mean+SEM.

FIG. 8 shows assay workflows. C2C12 myocytes were used in all assays, except in the Glut4 translocation assay where L6 rat myocytes were used. ICW, In-Cell Western. Also see Methods.

FIG. 9 shows ligand activity profiles show a wide range of variance required for statistical analyses. A) Compounds were profiled in 384-well format as described in FIG. 2, including 22 novel GCs, PF802, Dex, and RU486 at 10 μM doses dispensed by a 100 nl pin-tool robot. For native gene expression, C2C12 myotubes were treated compounds for 24 h. For MMTV-Luc assay, steroid-deprived 293T cells were co-transfected with a GR expression plasmid and an MMTV-driven luciferase reporter plasmid. The next day, cells were treated with compounds for 24 hrs. Each point represents the average of triplicate biological measurements for a separate compound. B) AR Luc assay development. Steroid-deprived 293T cells were transfected with AR expression plasmid and an ARR3-tk-driven luciferase reporter. The next day cells were treated with increasing doses of the indicated compounds. Data shown are mean+SD of triplicate measurements. C) AR-Luc assay identified 5 GCs with some AR activity. D) Ligand-dependent AR activity profiles do not correlate with protein synthesis.

FIG. 10 shows biological activities of several tested compounds. A) The effects of all tested compounds on Glut4 translocation to the myotube surface (y-axes) versus mitochondrial potential and chymotrypsin-like proteasomal activity i.e. protein degradation (x-axes). The Pearson correlation coefficient, r, and its associated p-value is indicated. B) Activity profiles of C11-substituted compounds in the indicated assays. C) Effects of all tested compounds on the nuclear translocation of GR (x-axes) versus protein degradation, mitochondrial potential and protein synthesis (y-axes). D) Effects of C11-substituted compounds on the nuclear translocation of GR (x-axes) versus Glut4 translocation, protein synthesis, mitochondrial potential and protein degradation (y-axes).

FIG. 11 shows activities of several texted compounds from further assays. A) The effects of all tested compounds on Glut4 translocation to the myotube surface (x-axes) versus the indicated mRNA levels (y-axes). The squared Pearson correlation coefficient, r² is indicated. B) The effects of all tested compounds on GR interaction with the PELP1_446 peptide (y-axes) versus the indicated mRNA levels (x-axes). C) The fractions of GC-regulated phenotype and mRNA level variance.

FIG. 12 shows activities of several compounds from additional assays. A) The effects of all tested compounds on IL-1β-induced IL-6 production (x-axes) versus GR interaction with the indicated peptides. B-C) The effects of all tested compounds on the indicated GC-regulated phenotypes (x-axes) versus GR interaction with the CHD9_1023 and CENPR_1 peptides (y-axes). D-F) COC plots. Each point represents the Pearson correlation coefficient, r, between a distinct gene expression or GR-peptide interaction assay with assays indicated on the x and y axes. G) The effects of all tested compounds on IL-1β-induced IL-6 production (y-axes) versus insulin-induced protein synthesis. H) Dose-dependent transactivation of the MMTV-Luc reporter in 293T treated with compounds SR11466 or SR16024 alone (i.e. agonist mode; solid lines), or in combination with Dex (i.e. antagonist mode; dashed lines). I) Inhibition of IL-6 by SR11466 was reversed by RU486. IL-β-induced IL-6 production by A549 cells treated with the indicated compounds was compared by AlphaLISA. Dex inhibits SR16024-dependent protein synthesis in C2C12 myotubes. K) Changes in the body weights of C57BL/6 male mice treated with DMSO vehicle, 10 mg/kg Dex or SR16024, or 50 mg/kg SR11466, were compared after an overnight fast. L) Dex and SR11466 increased the rate of glucose production, unlike SR16024. Mice in panel K were subjected to a lactate-tolerance test (LTT).

FIG. 13 shows modulator types, EC50 and IC50 values of various GC compounds on HepG2 cells. a Agonist: efficacy is higher than 90% in both agonist and antagonist modes; Partial agonist: efficacy is between 20% and 90% in agonist mode, and higher than 30% in antagonist mode; Antagonist: efficacy is lower than 20% in agonist mode, and lower than 30% in antagonist mode; NA: No Activity, efficacy does not fall into above three categories; b ND: Not Determined; c an antagonist at 10 μM dose.

FIG. 14 shows that a number of GC compounds have a better profile on protein synthesis (as indicated by the value of X axis) compared to dexamethasone (bottom line), while a subset are either neutral or anabolic (top line, vehicle). The value of Y axis indicates relative fluorescent units.

FIG. 15 shows results from further compound profiling studies. A) Each dot represents the effects of a compound in the indicated assays at 10 uM. N=4-6 from two different experiments. Data are normalized to vehicle (100%) and dex (0%) except for GILZ mRNA, where Dex is 100%. C) SR11466 is efficacious at inhibiting IL-6 secretion from A549 cells. C-E). Comparison of lead compounds with Dex and PF802, which was in clinical trials but did not progress These are the same data shown in B. C) Protein synthesis was measured in differentiated myotubes that were starved overnight and given GCs, and then stimulated for 1 hr the next day with insulin. D) Mitochondrial activity was measured in myoblasts using high content imaging with mitotracker dye after 24 treatment. New amino acid incorporation was measured with LICOR in cell western for puromycin (SunSet assay). E) top, MMTV-luciferase reporter data. N=3, mean+SEM. Bottom, Mineralization of primary human OBs was assayed after two weeks of differentiation from MSC and two weeks of mineralization. N=4, mean+SEM, 2-way ANOVA.

### DETAILED DESCRIPTION OF THE INVENTION

Glucocorticoids are well known and are frequently used for the treatment of acute and chronic inflammatory diseases, e.g., asthma, rheumatoid arthritis, inflammatory bowel diseases, multiple sclerosis and atopic dermatitis. Despite their broad therapeutic spectrum and superior therapeutic effects, long term systemic and local therapies with glucocorticoids are restricted due to side-effects. The most common side-effects related with systemic and topical application of a glucocorticoid are metabolic effects, including cachexia and muscle atrophy. Other side effects of GC treatment include suppression of HPA axis and the risk of induction of secondary adrenal suppression, induced gluconeogenesis, induced amino acid degradation, changes in electrolyte concentration, changes in lipid metabolism, growth retardation, osteoporosis, skin effects, including impaired wound healing, and skin thinning.

The present invention provides methods of preventing or suppressing side-effects, e.g., cachexia in skeletal muscle, that are associated with glucocorticoid treatment in mammalian subjects. The present invention is predicated in part on the studies undertaken by the inventors to understand the molecular basis of the various phenotypic activities of glucocorticoids and to identify analog GC compounds that are anabolic. Specifically, the inventors generated quantitative, statistically robust bioassays in myotubes and characterized a set of GCs designed to perturb the glucocorticoid receptor with several distinct structural mechanisms. As exemplified herein, the GC ligands displayed a full range of variance across the skeletal muscle bioassays, allowing the inventors to identify ligand specific gene expression patterns that were highly predictive for their effects on glucose disposal and protein balance. In vivo validation reveals that the ligand class analysis developed by the inventors can tie chemical and receptor structure to specific transcriptional signaling outcomes that define glucocorticoid action in skeletal muscle. Importantly, the profiling platform successfully predicted dissociated activity of the ligands, allowing the inventors to identify GC compounds that have strong anti-inflammatory activity without causing muscle atrophy and compounds that can block LPS-induced cachexia.

Employing the dissociated GCs with improved activities in skeletal muscle as described herein, the present invention provides methods for treating inflammatory conditions or autoimmune diseases. The invention also provides methods for ameliorating an undesired inflammation in subjects who are undergoing chemotherapy. Some other therapeutic methods of the invention are directed to treating muscular dystrophy, cachexia and related conditions such as muscle wasting and muscle weakness. The invention also provides kits for carrying out the various therapeutic regimens described herein. The following sections provide more detailed guidance for practicing the invention.

The practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

An “antagonist” according to the invention can be a substance, which binds to a GR and thereby prevents binding of an endogenous or exogenous agonist. An “agonist” according to the invention can be an endogenous or exogenous glucocorticoid, which induces by binding to a GR known glucocorticoid mediated cellular effects. A “partial agonist” according to the invention is a substance binding to a GR and displaying agonistic as well as antagonistic activity.

As used herein, a combination therapy refers to treatment of a disorder or disease with a GC compound described herein in conjuction with a known agent for treating the same or a different condition in a subject. Administration of the two agents can be concurrent or sequential.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, a “patient” may be interchangeable with “subject” or “individual” and means an animal, which may be a human or non-human animal, in need of treatment. Non-human animals may include dogs, cats, horses, cows, pigs, sheep, and the like.

Cachexia, or wasting syndrome, is loss of weight, muscle atrophy, fatigue, weakness and significant loss of appetite in someone who is not actively trying to lose weight. It can be a sign of various underlying disorders; when a patient presents with cachexia, a doctor will generally consider the possibility of cancer, certain infectious diseases (e.g. tuberculosis, AIDS), and some autoimmune disorders, or addiction to drugs such as amphetamines or cocaine, chronic alcoholism and cirrhosis of the liver. Cachexia physically weakens patients to a state of immobility stemming from loss of appetite, asthenia, and anemia, and response to standard treatment is usually poor. See, e.g., Lainscak et al., Curr Opin Support Palliat Care 1: 299-305, 2007; and Bossola et al., Expert Opin Investig Drugs 16: 241-53, 2007.

Dissociated glucocorticoids refer to steroid compounds that can dissociate the transactivation function of glucocorticoids (GCs) from their transrepression function. GCs are mainly used to suppress disease-related inflammation and are widely used for the treatment of many inflammatory diseases including asthma and arthritis. However, GCs are also associated with debilitating side effects that place limitations on the long-term use of these drugs. GCs exert their effects by binding and activating the GC receptor (GR). The activated receptor then binds GC response elements (GREs) in the promoter of genes, and activates transcription (transactivation) or interferes with the activation of transcription by inhibiting the transactivating function of other transcription factors, such as AP-1 and NF-kB (transrepression). Transrepression is believed to be responsible for the majority of the beneficial anti-inflammatory effects of GCs, whereas transactivation is believed to play a bigger role in the unwanted side effects of GCs. By dissociating the transactivation function of GCs from the transrepression function to reduce side effects, dissociated glucocorticoids can allow more effective treatments for patients who require long-term suppression of inflammation. A well-known example of dissociated glucocorticoids is compound PF802 as described in Hu et al., Endocrinology 152, 3123-3134, 2011.

The phrase “therapeutically effective amount” as used herein means that amount of a compound, material, or composition comprising a compound of the present invention that is effective for producing some desired therapeutic effect, e.g., treating (i.e., preventing and/or ameliorating) inflammation in a subject, at a reasonable benefit/risk ratio applicable to any medical treatment. The therapeutically effective amount should be sufficient to reduce or eliminate at least one symptom. One of skill in the art recognizes that an amount may be considered therapeutically effective even if the cancer is not totally eradicated but improved partially.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Mammals may be referred to as “patients” or “subjects” or “individuals”.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

Administration “in conjunction with” one or more other therapeutic agents includes simultaneous (concurrent) and consecutive administration in any order.

The term “contacting” has its normal meaning and refers to combining two or more agents (e.g., polypeptides or small molecule compounds) or combining agents and cells. Contacting can occur in vitro, e.g., combining two or more agents or combining an agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

Inflammatory disorders are diseases caused by abnormally regulated inflammatory response, e.g., rheumatoid arthritis, hay fever, and atherosclerosis. Inflammation or inflammatory response refers to an innate immune response that occurs when tissues are injured by bacteria, trauma, toxins, heat, or any other cause. The damaged tissue releases compounds including histamine, bradykinin, and serotonin. Inflammation includes both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils.

The term “subject” for purposes of treatment refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., an inflammatory disorder), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of an inflammatory disease or disorder associated with miRNA, a therapeutic agent may directly decrease the pathology of the disease, or render the disease more susceptible to treatment by other therapeutic agents.

The present invention provides various therapeutic uses of a number of dissociated GC compounds that were found to have anabolic effects in skeletal muscle. In general, these GC compounds share a structure represented by Formula I,

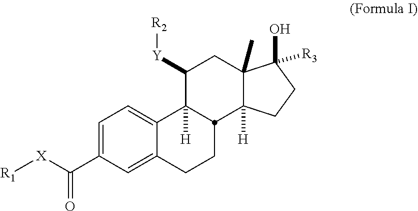

wherein X and Y are each independently O or NR; R₁=aryl, substituted aryl, pyridine, CN, OR, halogen, or Rₙ; R₂=H, C1-C3 alkyl or alkenyl, aryl, heteroaryl, halo, benzyl, halogen, CN, OR, or Rₙ; and R₃=C1-C3 alkyl, alkenyl, alkynyl, or Rₙ; and wherein n=0-3, R=H, C1-C4 alkyl, or haloalkyl. In some embodiments, the GC compound has a structure shown in Formula I, wherein X=NH, Y=O, R₁=pyridine, while R₂ is not methyl, ethyl, benzyl, 3-chlorobenzyl, 4-chlobenzyl or carbonate.

In some embodiments, the dissociated GC compounds used in invention have a structure as defined in Formula II (X=NH, Y=O, R₂=H, R₃=

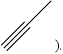

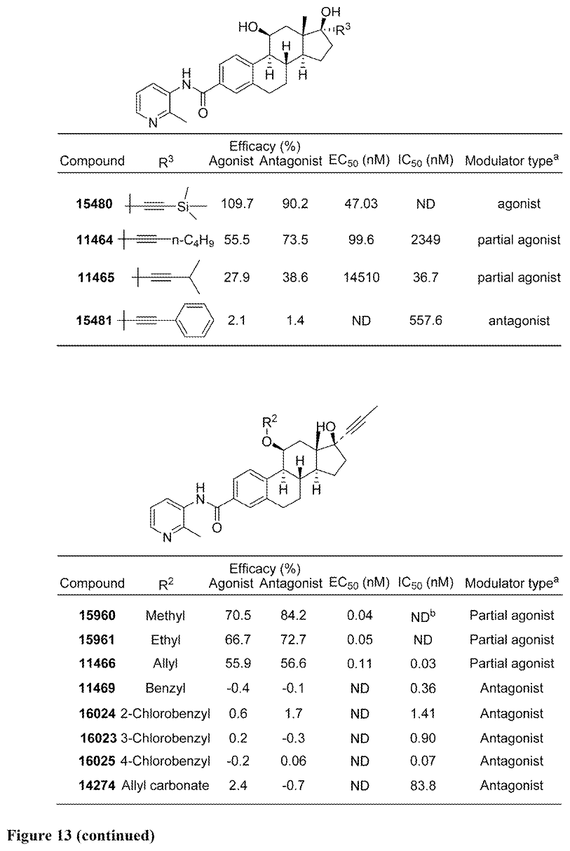

Examples of such compounds with different R₁ group, Compounds 15418, 15420, 15438, 15419, 15421, 15439 and 11461, are shown in FIG. 13. Also shown in the figure are their activities in HepG2 cells. In some embodiments, the dissociated GC compounds used in invention have a structure as defined in Formula III (X=NH, Y=O, R₁=pyridine, R₂=H). Structures and activities of examples of such compounds with different R₃, Compounds 15480, 11464, 11465 and 15481, are shown in FIG. 13. In some other embodiments, the dissociated GC compounds used in invention have a structure as defined in Formula IV (X=NH, Y=O, R₁=pyridine, R₃=

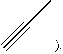

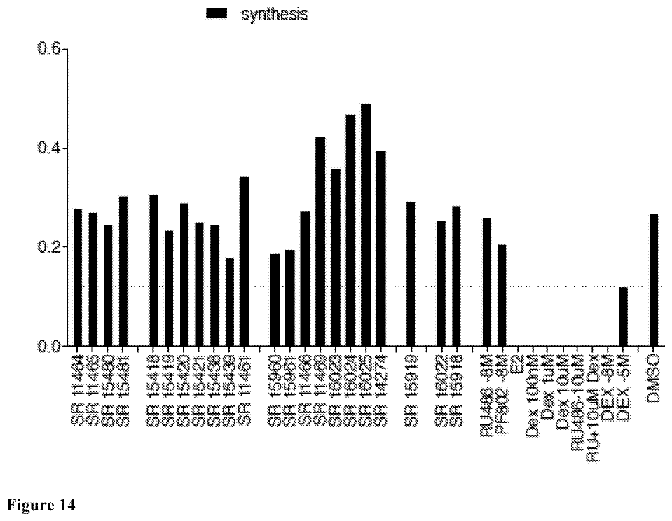

Structures and activities of examples of such compounds with different R₂, Compounds 15960, 15961, 11466, 11469, 16024, 16023, 16025 and 14274, are shown in FIG. 13. In some other embodiments, the dissociated GC compound used in invention is compound SR16022 or SR15918. The structures of these compounds and their activities are also shown in FIG. 13. As demonstrated by the experimental data described herein (e.g., FIGS. 13 and 14), these compounds, including antagonists and agonists, demonstrated improved activity in skeletal muscle. For example, compound SR11466 inhibited Glut4 translocation and inhibited IL-6 secretion as efficiently as dexamethasone in A549 lung cells, and better than PF802. Also as exemplified herein, compound SR16024 robustly stimulated protein synthesis, and inhibited proteasomal degradation as strongly as Dex. While known compounds PF802 and Dex strongly inhibited mitochondrial activity, the dissociated GC compounds described herein (e.g., SR11466 and SR16024) enhanced it. Some of the dissociated GC compounds also showed favorable pharmacokinetics for in vivo dosing, inhibited LPS-induced stimulation of TNFα in mice, and were protective against loss of lean mass following a larger dose of LPS.

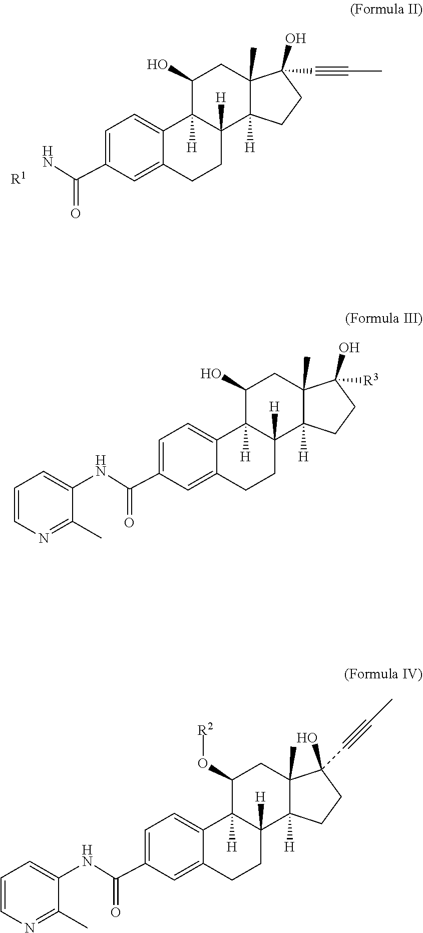

As provided in the invention, the dissociated GC compounds with anabolic effects in skeletal muscle as described herein can be employed in various therapeutic applications. Due to their improved side-effect profiles, these GCs provide better options than other known GCs in clinical settings where GCs are prescribed. Thus, in some embodiments, the invention provides methods of using these compounds to treat undesired inflammation, e.g., in inflammatory disorders or autoimmune diseases. Some of these methods are directed to treating or ameliorating an undesired inflammation in subjects who are undergoing chemotherapy. Various inflammatory conditions or autoimmune diseases are suitable for treatment with the methods of the invention. Examples include respiratory diseases (e.g., asthma), allergic diseases (e.g., hay fever, edema, serum sickness, contact dermatitis, drug reaction, urticaria, bee stings, angioneurotic edema, and anaphylaxis), arthritis (e.g. rheumatoid arthritis and osteoarthritis), rheumatic carditis, rheumatic fever, connective tissue diseases (e.g. systemic sclerosis or systemic lupus erythematosus, dermatomyositis, polymyositis, or mixed connective tissues diseases, polychondritis, and Sjogren's syndrome), vascular diseases (e.g. polyarteritis nodosa and granulomatous polyarteritis), skin diseases (e.g. psoriasis, atopic dermatitis and eczema), gastrointestinal diseases (e.g. an inflammatory bowel disease like chronic ulcerative colitis, Crohn's disease, gastritis, or esophagitis), renal diseases (e.g. glomerulonephritis and interstitial nephritis), liver diseases (e.g. subacute hepatic necrosis, chronic active hepatitis, alcoholic hepatitis or non-alcoholic hepatitis of various origin like chronic infection with hepatitis B virus or the like, and liver cirrhosis), ocular diseases (e.g. keratitis, uveitis, iritis, conjunctivitis, blepharitis, choroiditis, and neuritis nervi optici), ear diseases (e.g. otitis extema and otitis media), cerebral edema, shock, neurological diseases (e.g. multiple sclerosis and acute encephalomyelitis), meningitis, myastenia gravis, seizure, malignancy (e.g., acute lymphocytic leukemia, lymphoma, breast cancer, or prostate cancer), idiopathic thromocytopenia, haemolytic anemia, organ transplantations (e.g. suppression of tissue rejection, graft versus host disease), antiemetic therapy, endocrine diseases (e.g. Thyroiditis and adrenal hyperplasia), Tendonitis (bursitis), cushing syndrome, and metabolic diseases (e.g. diabetes esp. type 2 diabetes or obesity).

In some other embodiments, the invention provides methods of employing the dissociated GC compounds in treating or meliorating symptoms associated with cachexia, e.g., muscular dystrophy, muscle wasting and muscle weakness. Cachexia, which may also be referred to as wasting syndrome, occurs when there is a loss of body mass that cannot be reversed by nutritional means. Cachexia physically weakens patients to a state of immobility stemming from loss of appetite, asthenia and anemia, and response to standard treatment is usually poor. Cachexia includes sarcopenia as a part of its pathology. Examples of symptoms of cachexia include weight loss, muscle atrophy, fatigue, weakness, and/or considerable appetite loss in an individual that is not actively seeking to lose weight. In particular aspects, the cachexia is the result of a primary pathology, such as given that even if the affected individual consumes more calories, there is loss of body mass. In specific cases, skeletal muscle depletion is a prognostic factor.

Cachexia is often seen in end-stage cancer, and in that context is called “cancer cachexia”. It was also prevalent in HIV patients before the advent of highly active anti-retroviral therapy (HAART) for that condition; now it is seen less frequently in those countries where such treatment is available. In those patients who have Congestive Heart Failure, there is also a cachectic syndrome. Also, a cachexia co-morbidity is seen in patients that have any of the range of illnesses classified as “COPD” (chronic obstructive pulmonary disease), particularly emphysema. Some severe cases of schizophrenia can present this condition where it is named vesanic cachexia (from vesania, a Latin term for insanity).

Any subjects with cachexia or related conditions can be treated with the methods of the invention. These include subjects who are suffering from a muscular dystrophy such as Duchenne muscular dystrophy or Becker muscular dystrophy. They also include various disorders that are related to muscular dystrophy, e.g., dystrophinopathies, sarcoglycanopathies, limb girdle muscular dystrophies, congenital muscular dystrophies, congenital myopathies, distal myopathies, and myotonic syndromes. The subjects further include ones who may have an underlying condition or an unknown cause that results in muscle wasting and/or muscle weakness. The underlying condition may be a catabolic condition. The underlying condition may be chronic kidney disease, diabetes, cancer, AIDS, and so forth. Muscle wasting and/or muscle weakness embodiments may arise in the context of the individual also having cachexia, or the individual may not also have cachexia. The muscle wasting and/or muscle weakness may be the result of age or it may be the result of an underlying medical condition. The muscle wasting and/or muscle weakness may manifest prior to or after the detection of other symptoms of the underlying medical condition. Muscle wasting and/or muscle weakness can be examined for by a variety of ways, including physical examination; sitting and standing tests; walking tests; measurement of body mass index; reflex tests; blood tests for muscle enzymes; CT scan; measurement of total body nitrogen; muscle biopsy; and/or electromyogram, for example.

In some embodiments, the methods are directed to treating subjects who are at risk of developing muscle wasting or at risk of developing cachexia. As used herein, the phrase “at risk for developing muscle wasting” refers to a subject that is at risk for having less than their normal level of strength or too little muscle or having loss in muscle, such as an individual that has an underlying medical condition with such a symptom or is elderly. As used herein, the phrase “at risk for having cachexia” refers to subjects who are predisposed to having cachexia because of past, present, or future factors. In particular embodiments, a subject at risk for having cachexia is one that has an underlying condition that is known to cause or be associated with cachexia as at least one symptom. The condition may or may not be chronic. In some embodiments, an underlying medical condition that is known to have cachexia as at least one symptom includes at least renal failure, cancer, AIDS, HIV infection, chronic obstructive lung disease (including emphysema), multiple sclerosis, congestive heart failure, tuberculosis, familial amyloid polyneuropathy, acrodynia, hormonal deficiency, metabolic acidosis, infectious disease, chronic pancreatitis, autoimmune disorder, celiac disease, Crohn's disease, electrolyte imbalance, Addison's disease, sepsis, burns, trauma, fever, long bone fracture, hyperthyroidism, prolonged steroid therapy, surgery, bone marrow transplant, atypical pneumonia, brucellosis, endocarditis, Hepatitis B, lung abscess, mastocytosis, paraneoplastic syndrome, polyarteritis nodosa, sarcoidosis, systemic lupus erythematosus, myositis, polymyositis, dematomyosytis, rheumatological diseases, autoimmune disease, collagen-vascular disease, visceral leishmaniasis, prolonged bed rest, and/or addiction to drugs, such as amphetamine, opiates, or barbiturates.

In some embodiments, subjects in need of treatment can be administered with a pharmaceutical composition containing a GC compound described herein in combination with another known agent for treating muscle wasting and/or muscle weakness and/or cachexia treatment. Examples of known cachexia treatment include anabolic steroids; drugs that mimic progesterone; BMS-945429 (also known as ALD518); Enobosarm; propranolol and etodolac; omega-3 fatty acids; medical marijuana, IGF-1; nutritional supplements and/or exercise. Also, various agents have been administered in attempts to retard or halt progressive cachexia in cancer patients. These agents include orexigenic agents (appetite stimulants), corticosteroids, cannabinoids, serotonin antagonists, prokinetic agents, androgens and anabolic agents, anticytokine agents, NSAIDs, and regulators of circadian rhythm.

The dissociated GC compounds and the other therapeutic agents disclosed herein can be administered directly to subjects in need of treatment. However, these therapeutic compounds are preferable administered to the subjects in pharmaceutical compositions which comprise the dissociated GC compound and/or other active agents along with a pharmaceutically acceptable carrier, diluent or excipient in unit dosage form. Accordingly, the invention provides pharmaceutical compositions comprising one or more of the dissociated GC compound compounds disclosed herein. The invention also provides a use of these dissociated GC compounds in the preparation of pharmaceutical compositions or medicaments for treating the above described diseases or medical disorders.

Pharmaceutically acceptable carriers are agents which are not biologically or otherwise undesirable. These agents can be administered to a subject along with a dissociated GC compound compound without causing any undesirable biological effects or interacting in a deleterious manner with any of the components of the pharmaceutical composition. The compositions can additionally contain other therapeutic agents that are suitable for treating inflammation. Pharmaceutically carriers enhance or stabilize the composition or facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration described herein. Additional guidance for selecting appropriate pharmaceutically acceptable carriers is provided in the art, e.g., Remington: The Science and Practice of Pharmacy, Mack Publishing Co., 20^{th} ed., 2000.

A pharmaceutical composition containing a dissociated GC compound compound described herein and/or other therapeutic agents can be administered by a variety of methods known in the art. The routes and/or modes of administration vary depending upon the desired results. In some embodiments, a dissociated GC compound described herein can be administered to the subject via systemic route, e.g., by injection. In some other embodiments, the compound is administered to the subject via local administration. Depending on the route of administration, the active therapeutic agent may be coated in a material to protect the compound from the action of acids and other natural conditions that may inactivate the agent. Conventional pharmaceutical practice may be employed to provide suitable formulations to administer such compositions to subjects. Any appropriate route of administration may be employed, for example, but not limited to, intravenous, parenteral, transcutaneous, subcutaneous, intramuscular, intracranial, intraorbital, intraventricular, intracapsular, intraspinal or oral administration. Depending on the specific conditions of the subject to be treated, either systemic or localized delivery of the therapeutic agents may be used in the treatment.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

The dissociated GC compounds for use in the methods of the invention should be administered to a subject in an amount that is sufficient to achieve the desired therapeutic effect (e.g., eliminating or ameliorating symptoms associated with undesired inflammation) in a subject in need thereof. Typically, a therapeutically effective dose or efficacious dose of the dissociated GC compound is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

The dissociated GC compound compounds and other therapeutic regimens described herein are usually administered to the subjects on multiple occasions. Intervals between single dosages can be daily, weekly, monthly or yearly. Dosage and frequency vary depending on the half-life of the dissociated GC compound compound and the other drugs in the subject. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regimen.

The dissociated GC compounds and pharmaceutical compositions described herein can be provided in kits for the intended therapeutic uses. Typically, the kits include a suitable container means which houses one or more dissociated GC compounds or a pharmaceutical composition described herein. The kits can include additional reagents that may be needed for performing the therapeutic methods. In some embodiments, the kits can contain additional agents that can be used in combination with the dissociated GC compounds for treating undesired inflammation or cachexia. In some embodiments, the kits can include an apparatus or a means for the diagnosis of the underlying diseases or conditions that are intended to be treated, muscle wasting and/or muscle weakness.

In various embodiments, the components of the kits can be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the composition, additional agent, and any other reagent containers in close confinement for commercial sale. Such containers may include injection or blow molded plastic containers into which the desired vials are retained. Typically, the kits of the invention can additionally contain a written instruction for using the various components in performing the therapeutic methods.

### EXAMPLES

The following examples are provided to further illustrate the invention but not to limit its scope. Other variants of the invention will be readily apparent to one of ordinary skill in the art and are encompassed by the appended claims.

### Example 1. Structure-Based Design Strategy for Dissociated Glucocorticoids

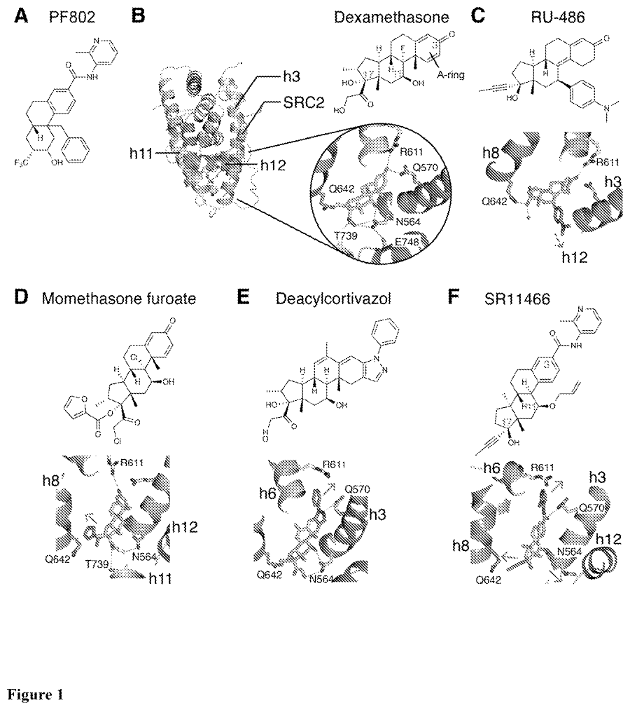

Structure-based design targeting GR has been hampered by the difficulty in obtaining crystal structures. By starting with a steroidal scaffold with known binding mode to GR, we can design a series of analogous ligands, with high assurance of how distinct chemical modifications will perturb receptor structure. We designed a series of glucocorticoids (GCs) to test the role of three different structural perturbations that are found in PF802 (Hu et al., Endocrinology 152, 3123-3134, 2011), a compound for which there was a pro-drug in clinical trials as a dissociated GC (Stock et al., Int J Rheum Dis.20: 960-970, 2017) (FIG. 1, A). The GR ligand binding domain (LBD) is comprised of 12 helices and a β-sheet, and the ligand binding pocket is dynamic in the absence of ligands. Full agonists such as Dex stabilize docking of the last helix, h12, across h3 and h11 to form one side of the coactivator-binding site, called Activation Function-2 (AF-2). This surface is the nucleating binding site for diverse transcriptional coregulators, which form protein complexes that can also interact with other receptor domains.

Steroids are compounds with 4 rings, lettered A-D. A ketone at the carbon-3 (C3) position of the A-ring forms hydrogen bonds with Arg and Asn residues, which are typically required for high affinity binding (FIG. 1, B). A hydroxyl at carbon-11 (C11) makes contacts with Asn564 in h3 that in turn stabilizes the active conformation of h12 by h-bonding to E748 (FIG. 1, B). The traditional route for generating nuclear receptor antagonists is to take an agonist and append a bulky side group that displaces h12 from the agonist position, typified by the dimethylanaline group attached at C11 in RU486 (FIG. 1, C). Substitutions at C3, as seen with the furoate group in momethasone (FIG. 1, D) or the propyne in RU486 target a small internal pocket to increase affinity, but can also subtly shift the ligand core, which we predict will alter the position of C11 substitutions that drive antagonism. While almost all GR ligands have a C3 ketone, the structure of deacylcortivazol showed that GR can accommodate extensions off the A-ring, which enters the solvent channel behind h3 (FIG. 1, E). We found that for the estrogen receptor, ligands that shift h3 alter the shape of the AF-2 surface and the ensemble of interacting proteins. This mechanism, for example, allows the phytoestrogen, resveratrol, to display strong anti-inflammatory activity, without inducing estrogenic proliferation due to reduced binding of Steroid Receptor Coactivator-3 to the AF-2 surface. Based on these observations, we hypothesize that chemistry targeting the solvent channel might produce novel biology for GCs and contribute to the dissociated effects of PF802.

PF802 contains a methylpyridinyl acetamide group attached at the C3 equivalent position that we predict interacts with the solvent channel behind h3, as well as a phenyl group to drive antagonism by perturbing h12, and a trifluoromethyl group attached at the equivalent position of C17 on a steroid (FIG. 1, A and B). In order to understand the how these different substitutions control activity, we generated a set of 22 related compounds based instead on a steroidal core (FIG. 1, F), which gives us good assurance of how the ligands are binding. These include a number of substitutions at C11 to modulate agonism/antagonism, different substitutions at C17, and methylpyridinyl acetamide at C3, where the position of methyl and nitrogen in the pyridine ring were altered. We published the synthesis and preliminary structure activity relationships for these compounds (Jin et al., Bioorg. & Med. Chem. Lett. 27, 347-353, 2017), demonstrating that they show a full range of graded activities from full agonist to full antagonist in luciferase assays, and for expression of canonical GR target genes, Fkbp5 and Pdk4, in mouse C2C12 myotubes.

### Example 2. Physiological GC Phenotyping in Skeletal Muscle

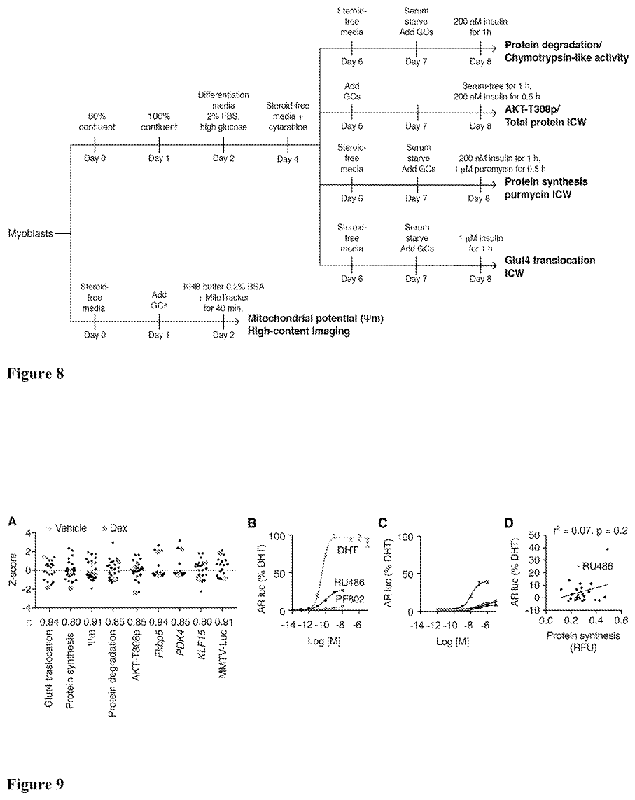

One of the barriers to understanding GC action in skeletal muscle is that effects on protein balance have required 50-100 μM Dex in vitro, despite a ˜5 nM Kd for GR. Here, we present a skeletal muscle profiling platform that allows visualization of Dex effects in the 1-10 nM dose range, based on the following key observations. We noticed that Dex strongly inhibits myoblast proliferation, and promotes fusion into myotubes, and thus we might expect the test compounds to have differential effects on differentiation, with agonists producing a more fully developed and mature network of myotubes, which are then subject to GC mediated atrophy of the myotubes. To avoid such effects on myogenesis, we switched myoblasts to differentiation media, and then on day 4, added 10 μM cytarabine, a chemotherapy agent that kills dividing cells, and then adding GCs on day 6 or 7 and completing assays in day 8 (FIG. 8).

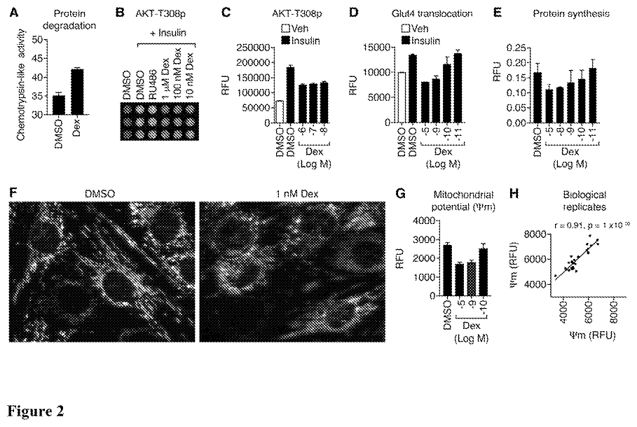

Physiologically, the effects of GCs on protein balance and Glut4 translocation vary in vivo depending upon the stressor. For example, GCs mediate atrophy in response to fasting but not exercise. We opted to assay GC effects on myotubes during serum starvation and followed by an insulin challenge, which combined with the cytarabine treatment, enabled Dex to show effects at 1-10 nM dosing on the insulin signaling pathway. To assess protein degradation, we measured chymotrypsin-like proteasomal activity (FIG. 2, A). The activating phosphorylation of AKT at Thr308 (pAKT) was measured using a LI-COR In-Cell Western assay (FIG. 2, B and C). The rate-limiting step in insulin-mediated glucose uptake is the translocation of the glucose transporter, Glut4, to the surface membrane, which we also measured by In-Cell Western without permeabilizing the myotubes (FIG. 2, D). Surface Sensing of Translation (SUnSET) (Schmidt et al., Nat Methods 6, 275-277, 2009) assay was used to measure protein synthesis by In-Cell Western, following treatment of myotubes with a low dose of puromycin, which incorporates into translating proteins, but at a low enough level to provide a readout for global protein synthesis (FIG. 2, E). Lastly, we developed a high-content imaging assay in myoblasts stained with a MitoTracker dye, so that individual mitochondrial networks could be visualized with adequate resolution to quantitate area, segmentation, branching, and fluorescence intensity which is a measure of mitochondrial membrane potential (ψm). The myoblasts display a reticulated mitochondrial network which was reduced by Dex, as was the brightness reflecting lower membrane potential (FIG. 2, F). 1 nM Dex was sufficient to produce the maximal loss of mitochondrial oxidative capacity (FIG. 2, G), and we observed low nM potencies for modulating pAKT, protein synthesis, and Glut4 translocation (FIG. 2, C and E), demonstrating that supraphysiological concentrations are not required under fasting conditions.

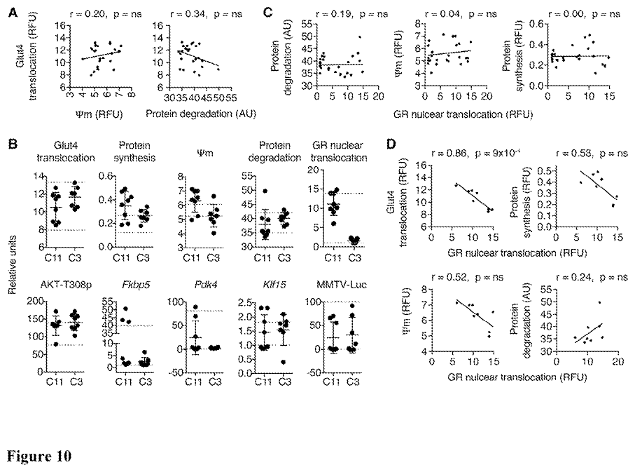

For inclusion in the statistical analyses, assays were required to show high reproducibility, with a Pearson correlation >0.8 between different experiments (FIG. 2, H; FIG. 9, A). In addition to the 22 new compounds, the 384-well compound plate includes RU486, PF802, and a dose curve of Dex. We also analyzed the effects of the compounds on expression of Fkbp5, Pdk4, and Klf15 mRNAs in C2C12 myotubes, and MMTV-luciferase and androgen receptor (AR) driven-luciferase activity in 293T cells. Fkbp5 and Pdk4 are among the most highly expressed GR target genes, while Klf15 is a known GR target gene that inhibits protein synthesis and stimulates expression of genes that facilitate protein degradation (Shimizu et al., Cell Metabol. 13, 170-182, 2011). We also analyzed the effects of compounds on nuclear translocation of GR using a quantitative HTS approach (Htun et al., Proc. Natl. Acad. Sci. USA 93, 4845-4850, 1996). To visualize the variance of the compounds, we expressed the data as Z score for each assay, in units of standard deviation (FIG. 9, A). For most of the assays, the compound-induced values were between the vehicle (DMSO) and Dex, such as the Glut4 translocation and protein degradation assays. However, we also identified some compounds that were full antagonists, and were anabolic for protein synthesis and mitochondrial oxidative capacity. We used an AR-luciferase assay to test for androgenic activity (FIG. 9, B). From this we identified a handful compounds with AR activity >5% (FIG. 9, C). While one compound showed partial AR agonist activity and promoted synthesis, the rest of the most anabolic compounds had no activity on AR-luc, and there was no correlation between the AR-luc data and protein synthesis (FIG. 9, D).

### Example 3. Ligand Class Analysis Reveals Different Signaling Patterns for Ligand Sub-Classes

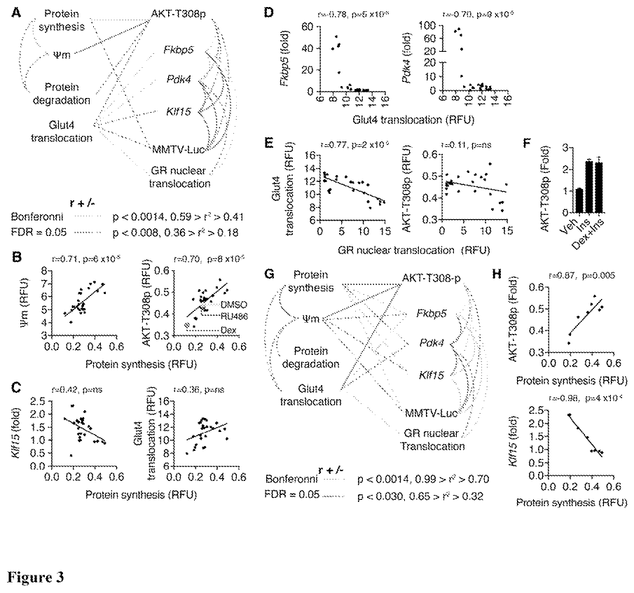

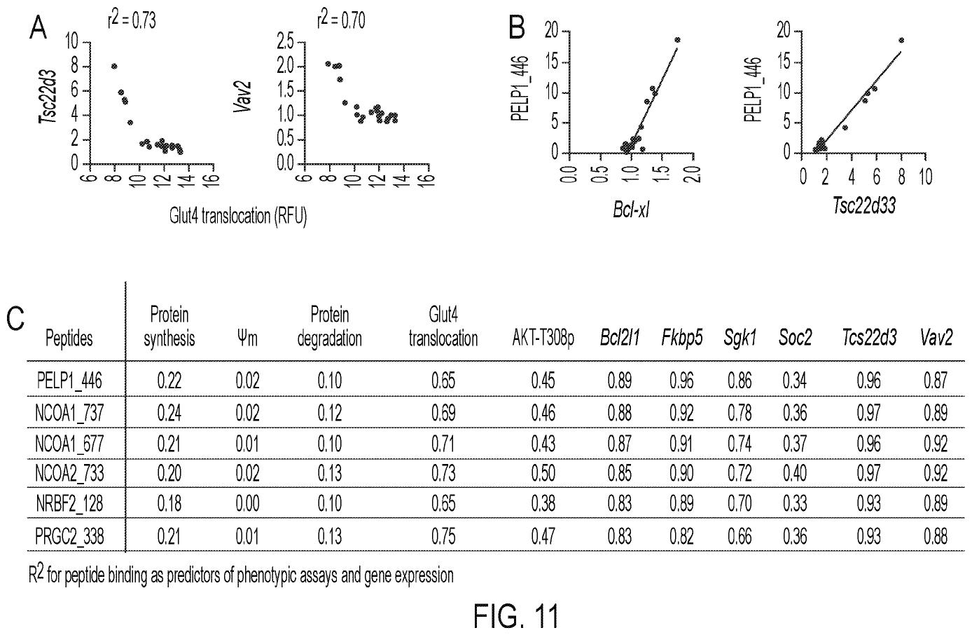

The wide range of variance in our assays enables us to identify ligand-induced signaling networks. We calculated Pearson correlations between the variables, and then defined two levels of significance using a strict Bonferonni correction for multiple hypothesis testing, and the less stringent Benjamini-Hockberg false discovery rate (FDR) procedure with 0.05 error rate. The correlation matrix and associate p-values are summarized in FIG. 3, A. Protein synthesis significantly correlated with ψm and pAKT (FIG. 3, B), but not Glut4 translocation or expression of these genes (FIG. 3, A and C). The compounds that stimulated protein synthesis above the vehicle also increased pAKT and ψm (FIG. 3, B). The effects of the ligands on Glut4 translocation were also dissociated from their effects on ψm and proteasomal degradation (FIG. 10, A), but instead were correlated with effects on Fkbp5 and Pdk4 gene expression and with nuclear translocation of GR (FIG. 3, A, D and E). The gene expression data showed non-linear relationships with the other variables, suggesting that a linear model underestimates the strength of these relationships, but demonstrates that the antagonists/partial agonists show a range of activities with minimal gene induction in skeletal muscle. The effects of all tested compounds on GR nuclear translocation was not correlated with their effects on pAKT, protein synthesis, degradation, or ψm (FIG. 3, E; FIG. 10, C). One possible explanation for the lack of correlation between nuclear translocation and pAKT would be that GR has non-genomic effects by binding directly to PI3K to inhibit its activation of AKT. However, insulin-mediated pAKT was not inhibited after 1 hr of Dex treatment, inconsistent with a non-genomic mechanism (FIG. 3, F). Further, the putative PI3K interaction motifs in GR are completely buried with the LBD and would require unfolding of the LBD for interaction, which is inconsistent with known properties of nuclear receptor LBDs.

We next examined the signaling patterns for subsets of compounds. Among the C17 substitutions, only the propyne bound GR with high affinity, so we stopped synthesis after producing a few of these and continued with the propyne for the other compounds (Jin et al., 2017; supra). We examined compounds differing in C11 substitutions and at compounds differing in the ring at C3. Examining the variance in the subsets shows a similar range of activity profiles, except for GR nuclear translocation and Fkbp5 and Pdk4 expression which showed lower activity in the C3- than in the C11-substituted compounds, despite several of the compounds strongly activating the MMTV-luciferase reporter (FIG. 10, B). As described below, it appears some of the effects of the ligands require little nuclear GR, while others are dosage sensitive.

To our surprise, there were no significant correlations between variables that define the activity profile of C3-substituted compounds. In contrast, the activity profiles of C11-substituted ligands indicate significant correlations among Glut4 translocation, protein synthesis, and mitochondrial oxidative capacity (FIG. 3, G). The r values for the correlations were much higher for the C11-substituted compounds than in the full ligand set. For example, pAKT showed improved prediction of protein synthesis (FIG. 3, H). By calculating r² from r, we can identify the percentage of the variance that is explained by the independent variable, so pAKT explains 49% of the variance in protein synthesis for the full set, and 68% of the variance in protein synthesis generated by the C11-substituted compounds (FIG. 3, H). Likewise, expression of Klf15 predicted 18% of the variance in synthesis with the full ligand set, but a remarkable 96% of the variance in the C11 data subset (FIG. 3, H). These findings demonstrate that modulating GR structure through the traditional C11 substitutions generates a transcriptional network with interconnected signaling outcomes for muscle phenotypes, while substitutions directed towards the solvent channel produce more dissociated outcomes. The full compound set displays a mixture of these properties, retaining some interconnected signaling.

### Example 4. Transcriptional Regulators and Coregulators of Ligand-Selective GR Function in Skeletal Muscle

To extend our analysis beyond the canonical GR target genes i.e. Fkbp5, Pdk4, and Klf15, we identified other target genes in myotubes by nascent RNA-seq. After 1 hr of treatment with 10 μM Dex, chromatin-associated RNA transcripts were extracted, sequenced, and analyzed to identify Dex-dependent transcripts representing primary GR target genes. We identified 501 differentially expressed genes, about ⅓ of which were previously identified in microarray experiments (Kuo et al., Proc. Natl. Acad. Sci. USA 109:11160-11165, 2012). We identified several novel canonical signaling pathways from nascent RNA data, including regulation of insulin receptor, mTOR, and ceramide signaling, as well as regulation of EIF4 and p70S6K signaling. We also identified putative upstream regulators with known roles in skeletal muscle such as HDAC4. For disease or functions annotations, Dex-regulated genes were implicated in muscle cancers, obesity, cell-cell contact, and morphology of nervous system, among others.

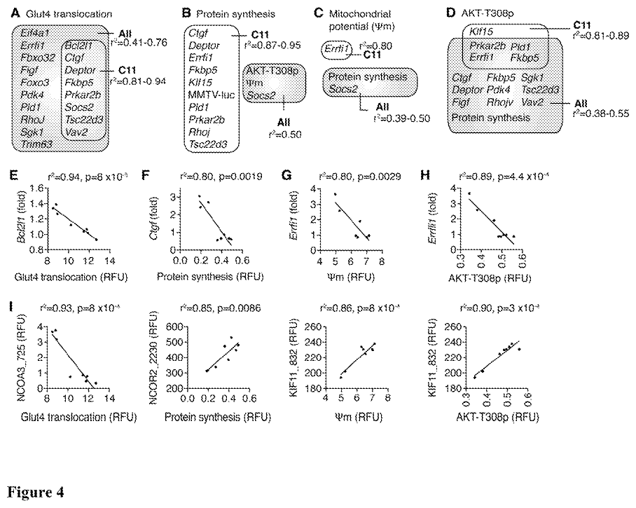

To develop quantitative assays for GR target genes, we used the nanoString nCounter for direct multiplexed transcript counting without PCR amplification (Geiss et al., Nat. Biotechnol. 26:317-325, 2008). Based on their nascent RNA-seq expression profiles and known roles as GR target genes, 29 genes were selected and assayed by nanoString nCounter in C2C12 myotubes treated with the 22 compounds (Table 1). Two different probes were tested against Pkd4 and Fkbp5. The probe pairs showed correlation >0.98 with each other. For Fkbp5, the nanoString probes correlated with the PCR data, r=0.93-0.94, while for Pdk4 the nanoString data correlated with the PCR data, r=0.83-0.84. As discussed further below, many of the genes were highly intercorrelated, so we opted for a strict Bonferonni p-value for multiple hypothesis testing. Expression of 18 of the genes significantly correlated with Glut4 translocation (FIG. 4, A-D), 8 of which showed r²>0.5. It is noteworthy that many of these significantly correlated genes did so with changes in gene expression that were 2-fold or less, such as the Bcl2l1 gene, which encodes for Bcl-xL, mitochondrial BCL-2 family member that is anti-apoptotic, but inhibits glucose metabolism and mitochondrial-mediated secretion of insulin by pancreatic β-cells. When we examined the C11- versus C3-substituted compounds, once again there were no significant predictors of Glut4 translocation among the C3-substituted compounds. Genes that met the Bonferonni-corrected p-value threshold are shown in the bigger box (FIG. 4, A). Only a subset of these genes shown in the inner box significantly predicted the variance in Glut4 translocation induced by C11-substituted compounds, but with much higher r² values compared to the full set of compounds (FIG. 4, A). It is remarkable that Bcl2l1 predicted 94% of the variance in Glut4 translocation for the C11-substituted compounds (FIG. 4, E).

TABLE 1
Genes tested as potential targets of the GC compounds

| Gene | PMID | Annotation |
|---|---|---|
| ACSS1 | 19187775 | Important for maintaining normal body temperature during fasting |
| | | and for energy homeostasis. Essential for energy expenditure |
| | | under ketogenic conditions (By similarity). Converts acetate to |
| | | acetyl-CoA so that it can be used for oxidation through the |
| | | tricarboxylic cycle to produce ATP and CO2. |
| BCL2L1 | 8358789 | The proteins encoded by BCL2L1 are located at the outer |
| | | mitochondrial membrane, and have been shown to regulate outer |
| | | mitochondrial membrane channel (VDAC) opening. VDAC |
| | | regulates mitochondrial membrane potential, and thus controls the |
| | | production of reactive oxygen species and release of cytochrome C |
| | | by mitochondria, both of which are the potent inducers of cell |
| | | apoptosis. |
| CTGF | 22129992 | CTGF has important roles in many biological processes, including |
| | | cell adhesion, migration, proliferation, angiogenesis, skeletal |
| | | development, and tissue wound repair, and is critically involved in |
| | | fibrotic disease |
| DDIT4 | 19297425 | DDIT4 acts as a negative regulator of mTOR, |
| DEPTOR | 28086984 | Overexpression of DEPTOR downregulates the activity of |
| | | mTORC1 and mTORC2 in vitro. mTORC1 and mTORC2 can |
| | | both inhibit DEPTOR through phosphorylation. |
| EIF4A1 | 14718385 | ATP-dependent RNA helicase which is a subunit of the eIF4F |
| | | complex involved in cap recognition and is required for mRNA |
| | | binding to ribosome |
| EIF4A2 | 14718385 | ATP-dependent RNA helicase involved in cap recognition and is |
| | | required for mRNA binding to ribosome. EIF4A unwinds RNA |
| | | secondary structures in the 5′-UTR of mRNAs. |
| ERRFI1 | 7641805 | Acts as a negative regulator for several EGFR family members. |
| | | Inhibits EGFR catalytic activity. Plays a role in modulating the |
| | | response to steroid hormones in the uterus. |
| FBXO32 | 11679633 | FBXO32 (also known as MAFbx or Atrogin-1) was originally |
| | | identified as a muscle-specific gene required for muscle atrophy |
| FIGF | 10449752 | c-fos-induced growth factor/vascular endothelial growth factor D |
| | | (Figf/Vegf-D) |
| FKBP5 | 29170369 | FKBP51 regulates AKT2-AS160 signaling and metabolic |
| | | function. |
| FOXO3 | 15109499 | Foxo Transcription Factors Induce the Atrophy-Related Ubiquitin |
| | | Ligase Atrogin-1 and Cause Skeletal Muscle Atrophy |
| IRS1 | 21135130 | coordinate skeletal muscle growth and metabolism via the Akt and |
| | | AMPK pathways. |
| KLF15 | 21284984 | is increased by fasting and decreased by feeding and insulin via |
| | | PI3K signalling. |
| MAP3K8 | 11919155 | Activates IkappaB kinases, and thus induce the nuclear production |
| | | of NF-kappaB. Inhibition of these signaling cascades elicits |
| | | muscle atrophy in vitro and in vivo. |
| NFKBIA | 28556540 | Increased NF-κB activity has been implicated in the pathogenesis |
| | | of insulin resistance and muscle atrophy. |
| Nr4a3 | 24065705 | Overexpression of Nor1 in mice results in an oxidative, high- |
| | | endurance phenotype with increased mitochondrial number and |
| | | DNA, elevated myoglobin, enhanced ATP production, and PGC- |
| | | 1α gene expression |
| PDK4 | 24520982 | Plays a pivotal role in control of metabolic flexibility in skeletal |
| | | muscle |
| PLD1 | 23915343 | PLD1 expression regulates muscle cell size via the activation of |
| | | mTOR signaling |
| PPP2R2C | 26507691 | A Ser/Thr phosphatases that Interacts with IRS-1 via mTOR |
| | | competing for serine residues on IRS-1 and thereby regulating the |
| | | phosphorylation status of IRS-1. |
| PRKAR2B | 19536287 | Disruption of protein kinase A in mice enhances healthy aging. |
| Pax7 | 15501225 | Pax-7 up-regulation inhibits myogenesis and cell cycle progression |
| | | in satellite cells |
| RHOJ | 28980871 | RhoJ is an endothelial cell-restricted Rho GTPase that mediates |
| | | vascular morphogenesis and is regulated by the transcription factor |
| | | ERG |
| SGK1 | 23161797 | (SGK1) regulates muscle mass maintenance via downregulation of |
| | | proteolysis and autophagy as well as increased protein synthesis |
| SOCS2 | 24352701 | SOCS2 negatively regulates growth hormone action in vitro and in |
| | | vivo |
| TRIM63 | 25760630 | Muscle-specific E3 ubiquitin ligases are involved in muscle |
| | | atrophy of cancer cachexia: an in vitro and in vivo study. |
| TSC22D3 | 20124407 | results indicate that GILZ and L-GILZ bind and regulate |
| | | MyoD/HDAC1 transcriptional activity, thus mediating the anti- |
| | | myogenic effect of GCs. |
| VAV2 | 31075957 | Rho GTPase-mediated key processes include the release of insulin |
| | | from pancreatic β cells, glucose uptake into skeletal muscle and |
| | | adipose tissue, and muscle mass regulation. |
| VEGFC | 27511834 | overexpression of VEGF-C induces weight gain and insulin |
| | | resistance in mice |

The ligand sets also showed different signaling patterns, where Socs2 was the only target gene predictor of protein synthesis or mitochondrial oxidative capacity for the full compound set, where protein synthesis correlated with pAKT and mitochondrial oxidative capacity (FIG. 4, B-C). For the C11-substituted compounds, a different set of target genes and assays predicted protein synthesis or mitochondrial oxidative capacity (FIG. 4, B-C), while the C3-substituted compounds had no significant predictors for synthesis or mitochondrial oxidative capacity. For example, the Ctgf gene showed a bifurcated pattern with the C11-substituted compounds, where the three compounds that induced its expression inhibited protein synthesis, while the anabolic compounds repressed its expression (FIG. 4, F). Expression of this gene correlates with disease severity in Duchenne muscular dystrophy, while hemizygous deletion improves function in a mouse model of muscular dystrophy. pAKT displayed a pattern that was a hybrid of those seen with Glut4 translocation versus synthesis (FIG. 4, D). The list of significant predictors for the C11-substituted compounds was largely a subset of the predictors for all compounds, except for Klf15, which was unique to the C11-substituted compounds (FIG. 4, D). Here again, the correlated activity profiles of the C11-substituted compounds displayed higher r² values compared to the full ligand set.

There were a number of genes that were predictive across multiple assays, which may reflect coordination of the signaling pathways that control glucose disposal and protein balance in skeletal muscle. For C11-substituted compounds, Errfi1/Mig6 expression was significantly correlated with protein synthesis (FIG. 4, B), mitochondrial oxidative capacity (FIG. 4, G), and pAKT (FIG. 4, H), and predicted Glut4 translocation for the full ligand set (FIG. 4, A). Errfi1 encodes an inhibitor of EGFR/ERBB2 signaling, and liver-specific knockout of Errfi1 increased phosphorylation of EGFR, ERK1/2, AKT, mTOR, JNK, and IRS-1, and induced insulin resistance. Fbxo32 encodes the atrogin-1 protein that is well known to mediate atrophy, but is widely assumed to do so only through protein degradation. Its ability to predict synthesis suggests that atrogin-1 may also contribute to atrophy by inhibiting synthesis. Fkbp5 encodes a scaffold protein that directly inhibits AKT by recruiting a phosphatase, a role that can influence both glucose disposal and protein synthesis. However, the high degree of covariance between many of the genes suggests that some of the associations may be correlative rather than causal influences, reflecting usage of a common set of transcriptional coregulators.

As a structural probe for ligand-selective receptor conformers, we assayed interaction of the recombinant GR LBD with 154 nuclear receptor-interacting peptides derived from transcriptional coregulators and other proteins using MARCoNI. With this larger set none of the predictors met the FDR statistical threshold for significance, but there were many predictors of GR action with high r values. Peptides from the Ncoal-3 gene family (also known as _{SRC1}-3) predicted glucose disposal, while NCOR1 and NCOR2/SMRT peptide binding predicted anabolic effects on protein synthesis, and there were also many strong predictors for the other GR skeletal muscle assays (FIG. 4, I).

For the combined gene expression and peptide interaction dataset, the genes and peptides were interspersed in different hierarchical clusters, highlighting that distinct structural perturbations drive expression of specific transcripts. The ligands clustered into three major clades: Cluster-1 contained Dex and the three most agonistic C11-substituted compounds; cluster-2a contained the rest of the C11-substituted compounds; and cluster-2b contained the remaining compounds including PF802 and the DMSO vehicle. The assays clustered into four major clades. Mitochondrial potential, protein synthesis and interaction with NCOR1 and NCOR2 peptides were clustered together. The effects of the ligands on Glut4 translocation was in the same major clade, but clustered tightly with Irs1 expression, pAKT, and interaction with a PELP1 peptide. Proteasomal degradation clustered in one of the major clades with interaction with a PRDM2 peptide, and expression of Syk, Vegfc, Klf15, Map3k8, and Nfkbia which encodes IκBα. Map3k8 activates IκBα, which has well described roles in regulating skeletal muscle atrophy, as does Klf15. The third major clade contains a large number of intercorrelated assays that were highly activated by the agonists in ligand cluster-1.

Principal components analysis revealed that 80% of the variance in the ligand activity profiles could be accounted for by effects on Glut4 translocation (PC1) and on GR interaction with an IKBB peptide (PC2), and up to 94% of the variance when GR interaction with an NCOR2 peptide (PC3) is included. The first two principal components were sufficient to separate the three ligand clusters driven by perturbing the receptor structure in different locations. Diabetic patients have reduced IKKβ expression in skeletal muscle, which correlates with the degree of insulin resistance. Given the high degree of collinearity among many of the assays, we used machine-learning techniques to identify minimal sets of predictors and performed perturbation studies described below to determine causality.

### Example 5. Machine Learning Defines a Minimal Set of Predictors for GC Effects in Skeletal Muscle

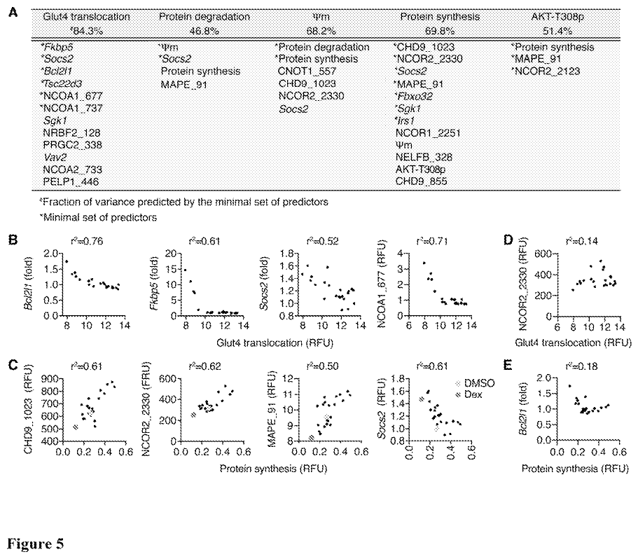

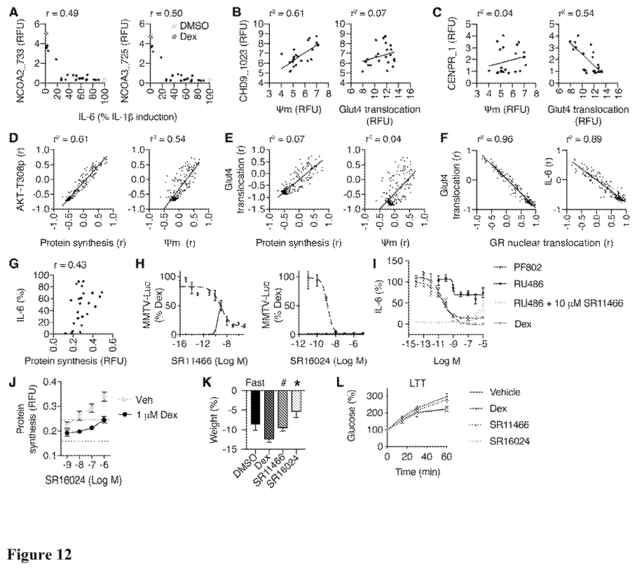

We initially used linear regression analysis for individual assays, but it is clear that most of the data is non-linear and many of the variables are highly intercorrelated, inspiring us to look for an approach that does not require linear data and can identify the best set of predictors. A decision tree-based classification algorithm, random forest, can be used to divide the data into subsets many times to create a forest of many classifications, and determine which classification best explains the data (Breiman., Machine Learning 45:5-32, 2001). This produces a rank order of features (i.e. the assays) that predict the dependent variable but does not provide a statistical cut-off that indicates which features are important. The Boruta algorithm resolves this issue by creating shuffled copies of the features and evaluates the degree to which random forest performs better on the real data compared to the shuffled data (Kursa, BMC Bioinformatics 15:8, 2014). Using these algorithms, we obtained a more restricted list of variables (FIG. 5, A). We were then able to determine which combination of variables had the greatest predictive power. For Glut4 translocation, 84% of the variance was explained by ligand-dependent modulation of 4 genes, Fkbp5, Blc2l1, Tsc22d3, and Socs2, plus the GR interaction profiles of two NCOA1/SRC1 peptides (FIG. 5, A-B; FIG. 11, A). Socs2 knockout mice are gigantic because Socs2 protein restrains growth hormone and IGF-1 signaling, although overexpression also causes gigantism, highlighting that it is highly dose-sensitive. Among the genes, Fkbp5, Blc2l1, Tsc22d3 all showed non-linear patterns with an inflection point, and both Blc2l1 and Socs2 showed induction <2-fold. To ask if very low levels of gene expression correlate with activity, we truncated the Glut4 data below the inflection point to remove the higher gene expression data points. Fkbp5, Blc2l1, Tsc22d3 and nuclear translocation still predicted Glut4 translocation, as did nuclear translocation (FIG. 11, B). This suggests that insulin-mediated glucose disposal can be fine-tuned by very small changes in GC-mediated gene expression.

We wondered if the peptide interactions predicted the expression levels of genes that in turn predicted effects on Glut4 translocation. We found that the 6 peptides were highly predictive for expression of genes that best predicted Glut4 translocation (FIG. 11, C-D). These peptides also showed poor predictive power for protein synthesis (r²=0.18-0.24), mitochondrial potential (r²=0.00-0.02) and protein degradation (r²=0.10-0.13), when compared to Glut4 translocation (r²=0.65-0.75) or pAKT (r²=0.38-0.48) (FIG. 11, C).

For protein degradation, 47% of the variance was explained by Socs2, mitochondrial oxidative capacity, protein synthesis, and GR interaction with a MAPE peptide (FIG. 5, A), suggesting that the effects of GR ligands on protein synthesis and degradation are coordinately regulated. Similarly, mitochondrial potential was predicted by protein synthesis (positively correlated) and degradation, a combination that constitutes the best predictive model, which explains 68% of the variance in mitochondrial potential (FIG. 5, A). This association between protein balance and mitochondrial function may reflect that protein balance is intimately tied to the energy requirements of the cell. The best model for protein synthesis, with ˜70% of the variance explained, included the expression levels of Socs2, Irs1, Sgk1, and Fbxo32, and GR interactions with CHD9, MAPE, and NCOR2/SMRT peptides (FIG. 5, A). Dex caused dissociation of these peptides from GR, while the anabolic compounds stimulated GR interactions with these peptides (FIG. 5, C). Recruitment of the NCOR2/SMRT peptide was selectively predictive, as it did not correlate with Glut4 translocation (FIG. 5, D), nor with the genes that best predicted Glut4 translocation (FIG. 11, C). The best model for pAKT included just GR interaction with MAPE and NCOR2/SMRT peptides, as well as protein synthesis, to account for 51% of the variance. Thus, Ligand Class Analysis can identify molecular features that predict different aspects of glucocorticoid biology and do so using statistically significant signaling relationships in what would typically be considered noise.

### Example 6. Perturbation Studies Define Causal Relationships Between Glucocorticoid-Regulated Genes and Skeletal Muscle Phenotypes

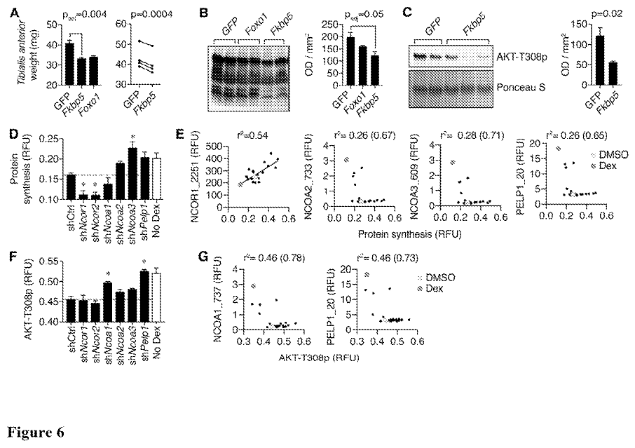

We performed gene perturbation studies to determine which correlations are due to causal relationships. We electroporated GFP and either Fkbp5 or Foxo1 expression plasmids into mice contralateral tibialis anterior muscles. Fkbp5 is expected to have pleiotropic effects based on our analyses (FIGS. 4 and 5), while Foxo1 is a positive control, overexpression of which reduces skeletal muscle size. Seven days later, mice were fasted overnight, treated for 1 hour with insulin, and injected during the last 30 minutes with puromycin for an in vivo SUnSET assay for insulin-induced protein synthesis. The Fkbp5 and Foxo1 injected TA muscles were approximately 10% smaller than control (FIG. 6, A-left). Electroporation with Foxo1 or Fkbp5 inhibited new protein synthesis (FIG. 6, B). We also repeated the electroporation with GFP and Fkbp5 expression plasmids into the contralateral TA muscle in the same mice for a within subjects' design and again found a significant decrease in muscle size (FIG. 6, A-right). Electroporation with Fkbp5 also inhibited insulin-induced pAKT in vivo (FIG. 6, C), suggesting a mechanism for atrophy via inhibition of insulin-induced protein synthesis.

From the peptide interaction data, we created lentiviral shRNA expression vectors for gene knockdown studies. Overall, the peptide interaction screen should be viewed as a structural assay for probing ligand-specific conformations, but some of them represent physiologically relevant interactions, including the NCOA1/SRC1, NCOA2/SRC2, NCOA6, NCOR1, NCOR2, and PELP1. Differentiated myotubes were transduced with shRNAs targeting these genes and assayed for Dex-mediated inhibition of protein synthesis and pAKT, in the context of nutrient deprivation and insulin challenge. Knockdown of Ncor1 or Ncor2/Smrt enhanced Dex-dependent inhibition of protein synthesis (FIG. 6, D), consistent with the peptide binding data showing that GR interaction with NCOR1 and NCOR2/SMRT corepressor peptides were stimulated by anabolic compounds (FIG. 5, C; FIG. 6, E). In contrast, knockdown of Ncoa6 and Pelp1 fully reversed the inhibitory effect of Dex, while knockdown of Ncoa2, but not Ncoa1, partially reversed this effect (FIG. 6, D). For NCOA2, NCOA6, and PELP1 interactions with GR, the r² for predicting protein synthesis was quite poor, due to a lack of any effect of the antagonists on the peptide interactions (FIG. 6, E). However, the strength of the interactions becomes apparent by focusing only on compounds that suppressed synthesis below the DMSO vehicle (FIG. 6, E; r² in parentheses). This demonstrates that the agonists utilize Ncoa2 and Pelp1 to inhibit protein synthesis, while the antagonists utilize Ncor1/Ncor2 to stimulate an anabolic effect. With pAKT, effects of Dex were significantly reversed by knockdown of Pelp1, but in this context, Ncoa1 but not Ncoa2 was required (FIG. 6, F-G). Knockout of Ncor1 during development has been reported to increase myofiber size by derepressing the ERRα transcription factor, so we repeated the knockdown of Ncor1 in myoblasts. We indeed found an increase in tube diameter after differentiation (FIG. 6, H), consistent with the pleiotropic effects of transcriptional coregulators in different physiological contexts. These data support a model where GR recruits specific coregulators to distinct subsets of target genes that have different effects on skeletal muscle metabolism and protein balance.

### Example 7. Discovery of Dissociated Glucocorticoids with Improved Skeletal Muscle Activity Profiles

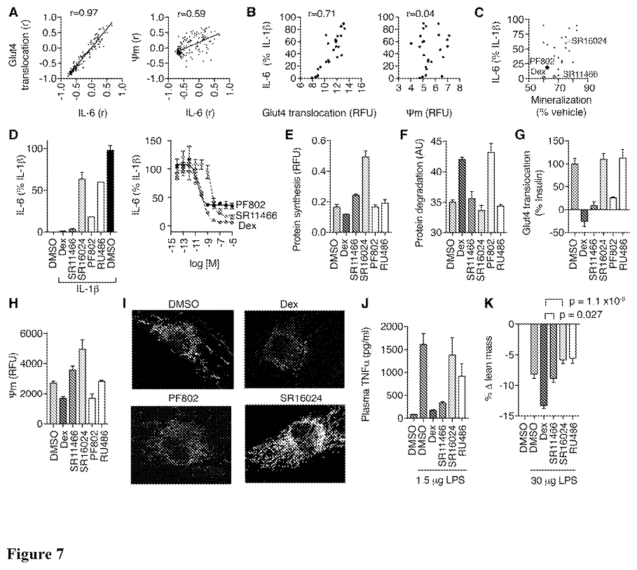

In order to understand whether effects on skeletal muscle could be dissociated from anti-inflammatory effects of the ligands, we profiled the compounds for suppression of IL1β-induced IL-6 secretion by A549 lung cells. Examining the correlations between assays can be used to identify dissociated assays that selectively correlate with a given phenotype, predicting discovery of dissociated ligands. In FIG. 7, Panel A, each point shows the Pearson correlation coefficient, r, for one specific gene expression profile or peptide interaction profile from the full ligand activity data set, where we compared the predictive power of the gene expression and peptide interaction assays for inhibiting IL-6 secretion, versus Glut4 translocation in the muscle cells. We call this a correlation of correlations (COC) plot. The COC plot shows that the assays with best predictive power for Glut4 translocation (Pearson correlation close to 1 or −1) are also the assays with the highest r for predicting IL-6 levels, demonstrating that similar GR conformations and other biophysical properties control the inhibition of IL-6 secretion and Glut4 translocation (FIG. 7, A). These include recruitment of NCOA2 and NCOA3 peptides, coactivators that we and others have shown to be required for GC-dependent suppression of inflammation in different cell types (FIG. 11, A). However, for the mitochondrial assay and IL-6, there are dissociated assays shown by points that are far out from the regression line in the COC plot (FIG. 7, A). For example, interaction of CHD9 with GR predicted mitochondrial activity but not Glut4 translocation (FIG. 11, B), while interaction with the CENPR peptide selectively predicted effects on Glut4 translocation (FIG. 11, C). The COC curves showed that the power of the assays to predict pAKT and protein synthesis or mitochondrial potential are correlated (FIG. 11, D) but dissociated from their ability to predict Glut4 translocation (FIG. 11, E). Assays that correlated with effects of the ligands on GR nuclear translocation were also highly correlated with Glut4 translocation and suppression of IL-6 secretion (FIG. 11, F). Therefore, the COC curves provide a way to identify common signaling mechanisms between different GR target tissues and signaling pathways. Examination of the ligand activity profiles reveals that we were unable to separate inhibition of IL-6 from inhibition of Glut4 translocation, but effects on IL-6 levels were fully dissociated from ligand-induced effects on mitochondrial oxidative capacity (FIG. 7, B), and to a lesser extent, protein synthesis (FIG. 11, G).

Our ligand profiling platform revealed two types of compounds with improved activity in skeletal muscle. SR11466 profiled as a partial agonist with an IC50 of 0.24 nM for GR-driven luciferase activity, while SR16024 profiled as a full antagonist, with an IC50 of 1 nM for displacing 10 nM Dex. As a further test for dissociation, the compounds were profiled in primary human osteoblasts for effects on osteogenesis during 4 weeks of differentiation from mesenchymal stem cells. Specifically, osteoblast mineralization was examined as follows. Human mesenchymal stem cells (MSCs) at passage 2 were maintained in growth media consisted of alpha-MEM (Life Technologies, 32561-037) supplemented with 17% FBS (Sigma-Aldrich, 12303C), 2 mM L-glutarnine (Life Technologies, 25030-081), and 100 units/mL penicillin/streptomycin (Life Technologies, 15140-122). Effect of glucocorticoid receptor modulators on osteogenic differentiation of MSCs was quantified using Alizarin Red S (Sigma-Aldrich, A5533) staining. Briefly, human MSCs were seeded in the middle 8 wells of 48-well plates at plating density of 10,000 cells per well. PBS (Life Technologies, 10010-031) was added to surrounding wells to reduce evaporation of media in the middle wells during prolonged incubation. 24 h later, growth media was replaced by osteogenic induction media (OIM) consisted of low-glucose DMEM (Life Technologies, 10567-014) supplemented with 10% FBS (Sigma-Aldrich. 12303C), 50 μg/ml L-ascorbic-2-phosphate (Sigma-Aldrich, A8960), 10 mM β-glyceroiphosphate (Sigma-Aldrich, G9891), and glucocorticoid modulators. OIM containing test compounds was replaced every week. At the end of 2 weeks, monolayers were washed with PBS (Life Technologies, 10010-031), incubated in Richard-Allan Scientific™ Neutral Buffered Formalin (10%) (Thermo Scientific, 5725) for 2 h at room temperature, washed with deionized water and stained with Alizarin Red S for 30 min at room temperature. Monolayers were then rinsed 3×with deionized water until clear, stain was then extracted with 10% (w/v) cetylpyridinium chloride (Sigma-Aldrich, C0732) in 10 mM sodium phosphate (Sigma, S5011 and S5136), pH 7.0 for 15 min at room temperature and the amount of extracted dye quantified spectroscopically at 562 nm. Spectroscopic analysis performed using a synergy™ NEO HTS Multi-Mode Microplate Reader (BioTek Instruments, Inc.).

Results from the osteoblast mineralization study are shown in FIG. 7, C. The data indicate that it is possible to find GCs with improved bone-sparing properties compared to Dex (FIG. 7, C). We further found that SR11466 inhibited IL-1β-induced IL-6 secretion by A549 lung cells as efficiently as Dex, and with more efficacy than PF802, with 4 nM potency, while SR16024 partially inhibited IL-6 secretion, similar to RU486 (FIG. 7, D). The inhibition of IL-6 production by SR11466 was reversed by RU486, thus validating an on-target mechanism of action. In the differentiated myotubes, SR16024 robustly stimulated protein synthesis, while SR11466 was slightly anabolic (FIG. 7, E). Dex reversed the anabolic effect of SR16024, inducing a right-shift in the dose curve, again demonstrating on-target mechanism of action. PF802 stimulated protein degradation as strongly as Dex, while SR11466 and SR16024 had no effect (FIG. 7, F). With respect to Glut4 translocation, both PF802 and SR11466 were slightly improved compared to Dex, while SR16024 showed no inhibition (FIG. 7, G). Lastly, PF802 and Dex strongly inhibited mitochondrial activity, while SR11466 and SR16024 enhanced it, with SR16024 doubling both protein synthesis and mitochondrial potential (FIG. 7, E and H-I).

SR11466 and SR16024 showed favorable pharmacokinetics for in vivo dosing, so we determined their effects on LPS-induced TNFα levels in mice. Like Dex, SR11466 strongly suppressed TNFα levels in the blood, while the full antagonist SR16024 was not inhibitory (FIG. 7, J). We also assessed loss of lean mass following a larger dose of LPS, which was significantly worsened by Dex (FIG. 7, K). SR11466 was significantly better than Dex, while SR16024 was protective.

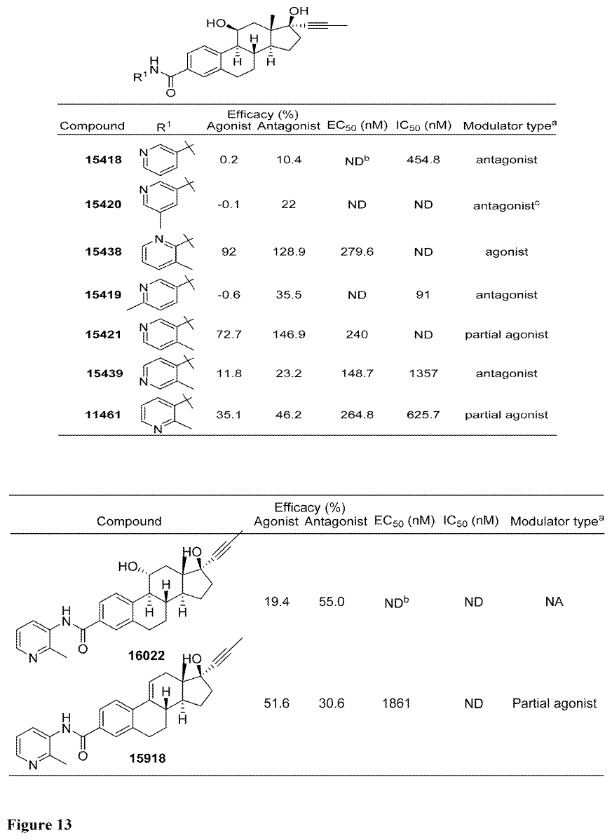

We were also interested in whether the compounds would show dissociation between Glut4 translocation in skeletal muscle and gluconeogenesis in the liver, but enhanced glucose production is typically studied after weeks or months of GC treatment. However, we reasoned that an overnight fast should enable detection of effects after a single dose. Again, SR11466 was significantly better than Dex at preserving body mass, while SR16024 showed a slight weight sparing effect after overnight fast (FIG. 12, K). The mice were then administered a lactate tolerance test, which showed that both Dex and SR11466 increased the rate of glucose production, while SR16024 did not (FIG. 12, L). Thus, profiling effects of GCs in the physiological context of nutrient deprivation allowed the discovery of two new types of glucocorticoids with improved profiles in skeletal muscle.

### Example 8. Further Compound Profiling Studies

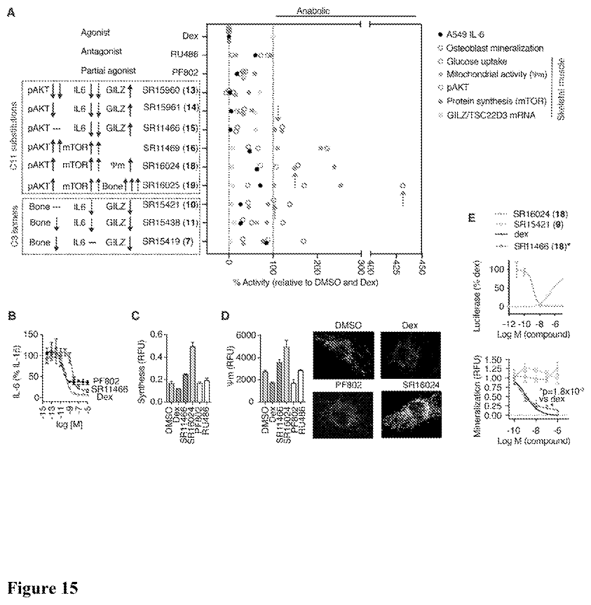

Additional studies were performed to assess activities of the various compounds identified from the previous profiling. Results from the studies are shown in FIG. 15. As shown in the figure, the antagonist RU-486 reversed most of the effects of dex, but still inhibited mineralization and mitochondrial activity, while compound PF-802 profiled as a partial agonist (FIG. 15, A). The carbon-11 substitutions produced pM and single digit nM compounds, including a wide range of activities. Compounds SR15960, SR15961 and SR11466 were all strongly anti-inflammatory, fully inhibiting IL-6 secretion but showing highly divergent effects on pAKT, protein synthesis, and mitochondria. These compounds similarly induced GILZ gene expression. Compound SR11466 represents the first SGRM that is anti-inflammatory but muscle sparing, with modest improvements on mineralization, with an EC50 of 0.1 nM in a luciferase reporter assay. Compounds SR11469, SR16024 and SR16025 profiled as more antagonistic SGRMs, not activating GILZ and were weakly anti-inflammatory. Compound SR16025 more than quadrupled mineralization, while testosterone had no effect in this assay, and the compound is a weak AR antagonist. These compounds are the first known anabolic antagonists of GR, stimulating mitochondrial activity, pAKT, protein synthesis, and bone to varying degrees (FIG. 15, A).

Compound SR16024 was selected for in vivo testing due to its bone sparing and anabolic profile in mitochondria and 1.5 nM IC50 in the reporter assay. The carbon-3 (C3) isomers with different positions of the methyl and nitrogen in the pyridine ring showed highly divergent effects on suppression of IL-6 and bone, with compound SR15421 being anti-inflammatory but completely bone sparing on the mineralization assay (FIG. 15, A, E). This compound was not advanced into animals due to 240 nM EC50 and will be further optimized. The assay data is shown in FIG. 15, B-E.

In summary, these follow-up studies confirmed that several compounds displayed anabolic effects, e.g., SR11466, SR11469, SR16023, SR16024, SR16025, SR15421, SR15438 and SR15419. In addition, compound SR15421 was shown to be anti-inflammatory but completely bone sparing. Moreover, compound SR16025 was found to quadruple bone mineralization.

### ***

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method of treating an inflammatory condition or ameliorating symptoms of undesired inflammation in a subject, comprising administering to a subject in need of treatment a pharmaceutical composition comprising a therapeutically effective amount of a compound of Formula I, thereby treating the inflammatory condition or ameliorating symptoms of undesired inflammation in the subject, 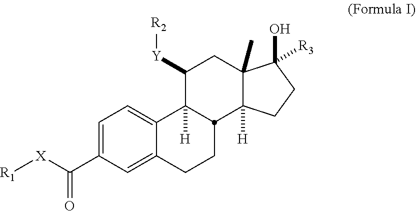
wherein X and Y are each independently O or NR;
R₁=aryl, substituted aryl, pyridine, CN, OR, halogen, or Rₙ;
R₂=H, C1-C3 alkyl or alkenyl, aryl, heteroaryl, halo, benzyl, halogen, CN, OR, or Rₙ;
R₃=C1-C3 alkyl, alkenyl, alkynyl, or Rₙ;
wherein n=0-3, R=H, C1-C4 alkyl, or haloalkyl.

2. The method of claim 1, wherein the compound is anabolic.

3. The method of claim 1, wherein the subject is afflicted with an inflammatory disorder or an autoimmune disease.

4. The method of claim 1, wherein if X=NH, Y=O, R₁=pyridine, then R₂ is not methyl, ethyl, benzyl, 3-chlorobenzyl, 4-chlobenzyl or carbonate.

5. The method of claim 1, wherein X=NH, Y=O, R₂=H, R₃=

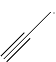

6. The method of claim 5, wherein the compound is compound 15418, 15420, 15438, 15419, 15421, 15439 or 11461 shown in FIG. 13.

7. The method of claim 1, wherein X=NH, Y=O, R₁=pyridine, R₂=H.

8. The method of claim 7, wherein the compound is compound 15480, 11464, 11465 or 15481 shown in FIG. 13.

9. The method of claim 1, wherein X=NH, Y=O, R₁=pyridine, R3=

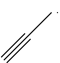

10. The method of claim 9, wherein the compound is compound 15960, 15961, 11466, 11469, 16024, 16023, 16025 or 14274 shown in FIG. 13.

11. The method of claim 1, wherein the compound is compound 16022 or 15918 shown in FIG. 13.

12. The method of claim 1, wherein the subject is also administered with an agent for chemotherapy.

13. The method of claim 12, wherein the agent for chemotherapy is administered to the subject prior to, simultaneously with or subsequent to administration with the compound.

14. The method of claim 1, wherein the subject is afflicted with cancer.

15. The method of claim 14, wherein the compound is any of the compounds shown in FIG. 13.

16. The method of claim 15, wherein the compound is 11466 or 16024.

17. A method for treating a muscular dystrophy or cachexia in a subject, comprising administering to the subject with a pharmaceutical composition comprising a therapeutically effective of a compound of Formula I, thereby treating the muscular dystrophy or cachexia in the subject, 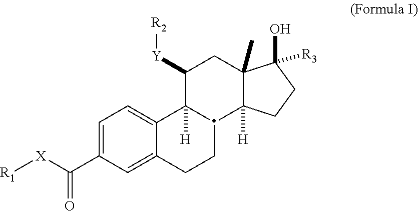
wherein X and Y are each independently O or NR;
R₁=aryl, substituted aryl, pyridine, CN, OR, halogen, or Rₙ;
R₂=C1-C3 alkyl or alkenyl, aryl, heteroaryl, halo, benzyl, halogen, CN, OR, or Rₙ;
R₃=C1-C3 alkyl, alkenyl, alkynyl, or Rₙ;
wherein n=0-3, R=H, C1-C4 alkyl, or haloalkyl.

18. The method of claim 17, wherein the compound is anabolic.

19. The method of claim 17, wherein if X=NH, Y=O, R₁=pyridine, then R₂ is not methyl, ethyl, benzyl, 3-chlorobenzyl, 4-chlobenzyl or carbonate.

20. The method of claim 17, wherein the compound is any of the compounds shown in FIG. 13.

21. The method of claim 20, wherein the compound is 11466 or 16024.

22. The method of claim 17, wherein the muscular dystrophy is Duchenne's muscular dystrophy.
